(19) United States
(12) Patent Application Publication
(10) Pub. No.: US 2025/0268902 A1
(43) Pub. Date: Aug. 28, 2025

(54) THERAPEUTIC METHODS

(71) Applicant: CeleCor Therapeutics, Inc., Del Mar, CA (US)

(72) Inventors: Barry S. Coller, New York, NY (US); C. Michael Gibson, Natick, MA (US); Arnoud W.J. van't Hof, Hattem (NL)

(73) Assignee: CeleCor Therapeutics, Inc., Del Mar, CA (US)

(21) Appl. No.: 18/277,364

(22) PCT Filed: Feb. 16, 2022
(86) PCT No.: PCT/US2022/016571

§ 371 Date: Aug. 15, 2023

Related U.S. Application Data
(60) Provisional application No. 63/150,301, filed on Feb. 17, 2021.

(51) Int. Cl. A61K 31/519; A61K 31/616; A61K 31/727; A61K 45/06; A61P 7/02

(52) U.S. Cl. CPC A61K 31/519; A61K 31/616; A61K 31/727; A61K 45/06; A61P 7/02

(57) ABSTRACT

The present disclosure relates to a method for treating or preventing a thrombotic disorder, comprising administering to a subject in need thereof an effective amount of the Compound (1) or a pharmaceutically acceptable salt thereof, at a dose in the range of 0.01-0.3 mg/kg, inclusive. The present disclosure further relates to compositions for use in such a method.

### RELATED APPLICATIONS

This application is a National Stage filing under 35 U.S.C. 371 of International PCT Application No. PCT/US2022/016571, filed Feb. 16, 2022, which claims priority under 35 U.S.C. § 119(e) to U.S. Provisional Application No. 63/150,301, filed Feb. 17, 2021, the contents of which are incorporated herein by reference in their entirety.

### BACKGROUND

A myocardial infarction (MI), commonly known as a heart attack, occurs when blood flow decreases or stops to a part of the heart, causing damage to the heart muscle. Worldwide, about 15.9 million myocardial infarctions occurred in 2015, and more than 3 million people had an ST elevation MI (STEMI). Of the people who die from MI, about half die within an hour of their first symptoms, and before they can reach the hospital. There is a direct relationship between the amount of time a heart artery is closed and the severity of the heart attack and odds of survival. Therefore, pre-hospital treatment is critical. In the past 40 years, in-hospital STEMI mortality has declined dramatically, but pre-hospital mortality remains unchanged.

Oral administration of P2Y12 antagonists administered along with aspirin at the first point of medical care, for example, in an ambulance, is not effective because these agents require several hours to achieve maximal effect in STEMI. Early intravenous administration of αIIβP3 (GPIIb/IIIa) antagonists improve clinical outcomes but require an initial bolus administration followed by continuous infusion.

Since transportation to a care facility such as a hospital can take longer than an hour, there is an urgent need for a rapidly-acting therapy that can be administered to MI patients to achieve high-grade inhibition of platelet function before they reach the care facility. Additionally, in order to reduce the risk of bleeding, it is desirable that the antiplatelet effect of the rapid-onset therapy diminish as the effects of commonly co-administered oral P2Y12 antagonists, such as current standard-of-care therapies, reach their maximal effects.

### SUMMARY

In one embodiment, provided herein is a method for treating or preventing a thrombotic disorder, comprising administering to a subject in need thereof an effective amount of the Compound (1):

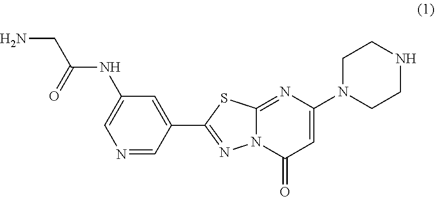 [SMILES: NCC(=O)Nc1cncc(-c2nn3c(=O)cc(N4CCNCC4)nc3s2)c1]

or a pharmaceutically acceptable salt thereof, at a dose in die e 0.01-0.3 mg/kg, inclusive. In certain embodiments, the thrombotic disorder is ST-elevated myocardial infarction (STEMI). In certain embodiments, Compound (1) is administered in the form of a solution comprising acetic acid, hydrochloric acid, glycerin, and water. In certain embodiments, Compound (1) is administered in the form of a lyophilized solid formulation.

### BRIEF DESCRIPTION OF THE DRAWINGS

FIG. 1 depicts inhibition of PAR1/PAR4-induced platelet aggregation (BASE Channel) measured with VerifyNow by Compound (1) (RUC-4) in STEMI patients treated with 1s Compound (I).

FIG. 2 depicts the mean simulated response-time relationship for Compound (I) by dose level for a 94.3 kg individual

FIG. 3 depicts the equivalence of VerifyNow and Light Transmission Aggregometry for measuring Compound (1) (RUC-4).

FIG. 4 depicts a schematic of platelet aggregation including relevant receptor-ligand interactions.

FIGS. 5A and 5B depicts inhibition of ADP-induced platelet aggregation by Compound (1) (RUC-4) as measured by Light Transmission Aggregometry in healthy subjects (5A), and subjects with stable coronary artery disease on aspirin (5B).

FIG. 6 depicts the pharmacodynamics of Compound (1) (RUC-4) and Ticagelor (P2Y12 inhibitor) and demonstrate their complementary effects in treating STEMI.

### DETAILED DESCRIPTION

RUC-4 is an inhibitor of the platelet αIIbβ3 receptor and is described in U.S. Pat. No. 9,303,044, which is incorporated herein by reference. In the present disclosure, RUC-4 is referred to as Compound (1). Mechanistically, RUC-4 inhibits ligand binding to aαIIbβ3 by binding to both the αIIb and β3 subunits and displacing the Mg2+ metal from the ion-dependent adhesion site (MIDAS) required for ligand binding; this locks the β3 subunit of the receptor in its inactive conformation. This may reduce the likelihood of thrombocytopenia because data indicate that much of the thrombocytopenia caused by the current aαIIbβ3 antagonists is due to the presence of antibodies to conformations of the receptor induced by the binding of the drugs. Thrombocytopenia is one of the potentially dangerous side effects of all of the current agents (˜0.5-2%). RUC-4 may also exhibit enhanced efficacy relative to blood thinners such as eptifibatide and tirofiban which induce a high-affinity binding conformation that may result in ligand binding and platelet aggregation as the drug levels decline.

In one aspect, provided herein is a method for treating or preventing a thrombotic disorder, comprising administering to a subject in need thereof an effective amount of the Compound (1):

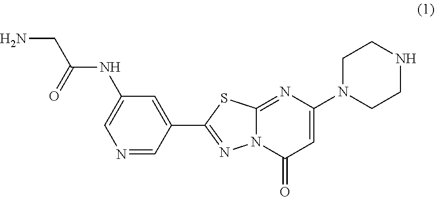 [SMILES: NCC(=O)Nc1cncc(-c2nn3c(=O)cc(N4CCNCC4)nc3s2)c1]

or a pharmaceutically acceptable salt thereof, at a dose in the range of 0.01-0.3 mg/kg, inclusive.

### Thrombotic Disorders

In certain embodiments, the thrombotic disorder is myocardial infarction (MI). In certain embodiments, the thrombotic disorder is ST-segment elevation myocardial infarction (STEMI), also referred to as ST-elevated myocardial infarction. In certain embodiments, the thrombotic disorder is non-ST-segment elevation myocardial infarction (NSTEMI). In certain embodiments, the thrombotic disorder is acute coronary syndrome (ACS). In certain embodiments, the thrombotic disorder is stroke. In certain embodiments, the thrombotic disorder is unstable angina. In certain embodiments, the thrombotic disorder is macrovascular or microvascular thrombosis caused by a diverse group of inciting events and affecting one or more organs or implanted or extracorporeal artificial materials, for example, retina, kidney, heart valve, vascular shunt or fistula, left-ventricular assist device, or intravascular cerebral coiled wire used to treat aneurysms.

### Dosage

In certain embodiments, the dose of Compound (1) or a pharmaceutically acceptable salt thereof is defined in terms of the amount of Compound (1) in free (i.e., non-salt) form. In certain embodiments, the dose is in the range of 0.01-0.3 mg/kg, inclusive. In certain embodiments, the dose is in the range of 0.05-0.3 mg/kg, inclusive. In certain embodiments, the dose is in the range of 0.1-0.3 mg/kg, inclusive. In certain embodiments, the dose is in the range of 0.15-0.3 mg/kg, inclusive. In certain embodiments, the dose is in the range of 0.2-0.3 mg/kg, inclusive. In certain embodiments, the dose is in the range of 0.25-0.3 mg/kg, inclusive. In certain embodiments, the dose is in the range of 0.01-0.2 mg/kg, inclusive. In certain embodiments, the dose is in the range of 0.05-0.2 mg/kg, inclusive. In certain embodiments, the dose is in the range of 0.1-0.2 mg/kg, inclusive. In certain embodiments, the dose is in the range of 0.15-0.2 mg/kg, inclusive. In certain embodiments, the dose is in the range of 0.01-0.15 mg/kg, inclusive. In certain embodiments, the dose is in the range of 0.05-0.15 mg/kg, inclusive. In certain embodiments, the dose is in the range of 0.1-0.15 mg/kg, inclusive. In certain embodiments, the dose is in the range of 0.07-0.075 mg/kg, inclusive. In certain embodiments, the dose is in the range of 0.08-0.085 mg/kg, inclusive. In certain embodiments, the dose is in the range of 0.09-0.095 mg/kg, inclusive. In certain embodiments, the dose is 0.07 mg/kg. In certain embodiments, the dose is 0.075 mg/kg. In certain embodiments, the dose is 0.08 mg/kg. In certain embodiments, the dose is 0.085 mg/kg. In certain embodiments, the dose is 0.09 mg/kg. In certain embodiments, the dose is 0.095 mg/kg. In certain embodiments, the dose is 0.10 mg/kg. In certain embodiments, the dose is 0.11 mg/kg. In certain embodiments, the dose is 0.12 mg/kg. In certain embodiments, the dose is 0.13 mg/kg. In certain embodiments, the dose is 0.14 mg/kg. In certain embodiments, the dose is 0.15 mg/kg.

In certain embodiments, the method comprising administering to the subject an acetate salt of Compound (1), designated Compound (1a). In certain embodiments, the acetate salt is a mono-acetate salt. In certain embodiments, the acetate salt is a di-acetate salt.

### Dose Selection

Several factors are considered in connection with dose selection. It is necessary to consistently achieve high grade inhibition of platelet aggregation (e.g., at least 80%) within 15 minutes of administration. It is necessary to minimize the risk of bleeding, particularly in persons of low body weight (e.g., elderly women) who have the highest risk of bleeding. It is also desirable for Compound (1) to persist, e.g., maintain at least 50% inhibition of platelet function, for up to 120 minutes.

However, the determination of antiplatelet effect attributable to Compound (1) may be confounded by the presence of other antiplatelet agents, such as aspirin, ticagrelor, and aαIIbβ3 antagonists other than Compound (1). Therefore, an assay is required that can differentiate the effects of Compound (1) from those of other antiplatelet agents.

Light transmission aggregometry (LTA) is considered the gold standard assay to assess the potency of antiplatelet agents, such as Compound (I), because of its association with clinical outcomes. As illustrated in FIG. 4, activators such as ADP, iso-TRAP, and Arachidonic Acid (AA) can be used to assess the differential effects of various platelet inhibitors. Compound (1) dramatically inhibits ADP and AA whereas aspirin does not significantly inhibit ADP or iso-TRAP induced LTA, and ticagrelor (and other P2Y12 inhibitors) inhibit the ADP induced LTA to various degrees, and slightly inhibits iso-TRAP induced LTA (depending on the dose administered). Addition of ADP+PAR-4 activating peptide completely inhibit the inhibition of platelet aggregation by P2Y12 inhibitors.

LTA may be difficult to perform during STEMI, as patients are being transported in an ambulance, or are being treated with percutaneous coronary intervention (PCI) in a cardiac catheterization laboratory away from the clinical lab. LTA requires significant manipulation of a blood sample and addition of reagents. As an alternative, rapid platelet function assays like VerifyNow can be used directly, using a blood sample without any manipulation. See, e.g., Joseph A. Jakubowski et al., Platelets, December 2011; 22(8): 619-625.

VerifyNow (VN) assays (Instrumentation Laboratories, Bedford, MA) are whole-blood, cartridge-based, and automated, and produce results within 15 minutes of blood draw. They are based on the agglutination of fibrinogen-coated beads by platelets activated by different agonists. Commercially available VN assays are designed to study the antiplatelet effects of aspirin (VN Aspirin cartridge; arachidonic acid activator) or P2Y12 antagonists (VN PRUTest cartridge and VN P2Y12 cartridge; ADP+PGE1 activator/inhibitor combination). The modified thrombin receptor activating peptide (iso-TRAP) is included in a separate reaction chamber (in addition to the ADP+PGE1 reaction chamber) in both the PRUTest and P2Y12 cartridges, and the P2Y12 cartridges further include a ‘BASE channel’ reaction chamber in which a PAR-1 activating peptide (iso-TRAP) at higher concentrations is combined with a PAR-4 activating peptide (PAR-4 AP) to achieve potent activation that can overcome the effects of P2Y12 antagonists.

VerifyNow ADP+PGE1, iso-TRAP, and base channel assays may be used to report Compound (1)-mediated inhibition in near real-time. VN assays can be used to help deconvolute antiplatelet effects of αIIbβ3 antagonists versus P2Y12 antagonists. Compound (1) dramatically inhibits all 3 VN assays, whereas aspirin does not inhibit the assays, and ticagrelor dramatically inhibits the ADP+PGE₁ assay, slightly inhibits the iso-TRAP assay, and does not inhibit the base channel assay. The BASE channel assay was used to monitor the pharmacodynamic effects of Compound (1) independent of any P2Y12 inhibitors.

### Compositions and Administration

In certain embodiments of the method, Compound (1) or a pharmaceutically acceptable salt thereof is administered in the form of a pharmaceutical composition comprising one or more carriers, diluents, or excipients. In certain embodiments, the pharmaceutical composition is a solution. In certain embodiments, the pharmaceutical composition is a microemulsion. In certain embodiments, the pharmaceutical composition is a suspension. In certain embodiments, the pharmaceutical composition is an emulsion. In certain embodiments, the pharmaceutical composition is a gel. In certain embodiments, the pharmaceutical composition is a slurry.

In certain embodiments, the pharmaceutical composition comprises a pharmaceutically acceptable salt of Compound (1). In certain particular embodiments, the pharmaceutically acceptable salt is an acetate salt, e.g., a mono-acetate salt or a di-acetate salt.

In certain embodiments, the pharmaceutical composition comprising Compound (1), a pharmaceutically acceptable salt thereof, is a solution. In certain embodiments, the concentration of Compound (1) or the pharmaceutically acceptable salt thereof (in the solution) is 0.01-100 mg/mL, inclusive. In certain embodiments, the concentration is 0.05-100 mg/mL, inclusive. In certain embodiments, the concentration is 0.1-100 mg/mL, inclusive. In certain embodiments, the concentration is 1-100 mg/mL, inclusive. In certain embodiments, the concentration is 10-100 mg/mL, inclusive. In certain embodiments, the concentration is 15-100 mg/mL, inclusive. In certain embodiments, the concentration is 20-100 mg/mL, inclusive. In certain embodiments, the concentration is 30-100 mg/mL, inclusive. In certain embodiments, the concentration is 40-100 mg/mL, inclusive. In certain embodiments, the concentration is 50-100 mg/mL, inclusive. In certain embodiments, the concentration is 0.05-50 mg/mL, inclusive. In certain embodiments, the concentration is 0.1-50 mg/mL, inclusive. In certain embodiments, the concentration is 1-50 mg/mL, inclusive. In certain embodiments, the concentration is 10-50 mg/mL, inclusive. In certain embodiments, the concentration is 15-50 mg/mL, inclusive. In certain embodiments, the concentration is 20-50 mg/mL, inclusive. In certain embodiments, the concentration is 30-50 mg/mL, inclusive. In certain embodiments, the concentration is 40-50 mg/mL, inclusive. In certain embodiments, the concentration is 0.05-25 mg/mL, inclusive. In certain embodiments, the concentration is 0.1-25 mg/mL, inclusive. In certain embodiments, the concentration is 1-25 mg/mL, inclusive. In certain embodiments, the concentration is 10-25 mg/mL, inclusive. In certain embodiments, the concentration is 15-25 mg/mL, inclusive. In certain embodiments, the concentration is 20-25 mg/mL, inclusive. In certain embodiments, the concentration is 0.05-10 mg/mL, inclusive. In certain embodiments, the concentration is 0.1-10 mg/mL, inclusive. In certain embodiments, the concentration is 1-10 mg/mL, inclusive.

In certain embodiments, the concentration of Compound (1), or a pharmaceutically acceptable salt thereof, in the solution is about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, or about 25 mg/mL.

In certain embodiments, Compound (1), or a pharmaceutically acceptable salt thereof, or a pharmaceutical composition as described herein, is administered to the subject by injection. In a particular embodiment, the injection is subcutaneous injection. In another particular embodiment, the site of injection includes the arm, leg, thigh, back, buttocks, and abdomen. In certain embodiments, administration by injection is performed using a pre-filled syringe, pen, or auto-injector. In certain embodiments, administration is self-administration by auto-injector.

In certain embodiments, the volume of the injection is 2 mL or less. In certain embodiments, the volume of the injection is 1 mL or less, e.g., about 0.9 mL, about 0.8 mL, about 0.7 mL, about 0.6 mL, about 0.5 mL, about 0.4 mL, about 0.3 mL, about 0.2 mL, or about 0.1 mL.

In certain embodiments, administration of Compound (1), or a pharmaceutically acceptable salt thereof, or a pharmaceutical composition as described herein, results in minimal injection site reaction (e.g., bruising, bleeding, swelling, and discomfort) in the subject at t=0, t=1 hour, t=1 day, t=3 days, or t=1 month post injection. In certain embodiments, the administration results in an injection site reaction that resolves in 30 days or less, e.g., within 25, 20, 15, 10, or 5 days.

In certain embodiments, the methods disclosed herein comprise a single administration (e.g., injection). In certain embodiments, the methods disclosed herein comprise multiple administrations (e.g., injections). For example, certain embodiments comprise administering a first dose of Compound (1), or a pharmaceutically acceptable salt thereof, or composition thereof, and optionally a second dose. A second dose is administered if such administration is desirable, advantageous, or necessary.

In certain embodiments, the dosage of a subsequent (e.g., second) administration is the same dose as the dosage of the first administration. In certain embodiments, the dosage of a subsequent (e.g., second) administration is different from the dosage of the first administration. Representative dosages, formulations, and concentrations are described herein.

### Combinations

In certain embodiments, the methods described herein further comprise administering to the subject an additional therapeutic compound (e.g., one or more additional therapeutic compounds). In certain embodiments, the additional therapeutic compound is a blood thinner. In certain embodiments, the additional therapeutic compound is heparin. In certain embodiments, the additional therapeutic compound is a P2Y12 inhibitor, e.g., ticagelor, prasugrel, or clopidogrel. In certain embodiments, the additional therapeutic compound is aspirin. In certain embodiments, the additional therapeutic compound is selected from cangrelor, warfarin, enoxaparin, fondaparinux, dalteparin, bivalirudin, hirudin, rivaroxaban, dabigatran, apixaban, and edoxaban.

In certain embodiments, the methods described herein further comprise administering to the subject a P2Y12 inhibitor and heparin. In certain embodiments, the methods described herein further comprise administering to the subject aspirin and heparin.

In certain embodiments, the additional therapeutic agent is administered to the subject prior to administration of Compound (1) or a pharmaceutically acceptable salt thereof, or a pharmaceutical composition as described herein. In certain embodiments, the additional therapeutic agent is administered to the subject following administration of Compound (1) or a pharmaceutically acceptable salt thereof, or a pharmaceutical composition as described herein. In certain embodiments, the additional therapeutic agent is administered to the subject concurrently with the administration of Compound (1) or a pharmaceutically acceptable salt thereof, or a pharmaceutical composition as described herein.

In certain embodiments of the methods described herein, the subject has been administered aspirin prior to the onset of symptoms, or after the onset of symptoms, but prior to administration of Compound (1) or a pharmaceutically acceptable salt thereof, or a pharmaceutical composition as described herein.

In certain embodiments of the methods described herein, the subject has been administered a P2Y12 inhibitor prior to the onset of symptoms, or after the onset of symptoms, but prior to administration of Compound (1) or a pharmaceutically acceptable salt thereof, or a pharmaceutical composition as described herein. In certain embodiments of the methods described herein, the subject has been administered aspirin and a P2Y12 inhibitor prior to the onset of symptoms, or after the onset of symptoms, but prior to administration of Compound (1) or a pharmaceutically acceptable salt thereof, or a pharmaceutical composition as described herein. In certain embodiments of the methods described herein, the subject is administered a P2Y12 inhibitor at the same time as administration of Compound (1) or a pharmaceutically acceptable salt thereof, or a pharmaceutical composition as described herein. In certain embodiments of the methods described herein, the subject is administered a P2Y12 inhibitor after the administration of Compound (1) or a pharmaceutically acceptable salt thereof, or a pharmaceutical composition as described herein.

### Administration

In certain embodiments of the method, the subject experiences one or more symptoms of the thrombotic disorder, and Compound (1) or a pharmaceutically acceptable salt thereof is administered within 6 hours of the onset of symptoms or the demonstration of ECG findings diagnostic of MI. In certain embodiments, Compound (1) or a pharmaceutically acceptable salt thereof is administered within 5 hours of the onset of symptoms. In certain embodiments, Compound (1) or a pharmaceutically acceptable salt thereof is administered within 4 hours of the onset of symptoms. In certain embodiments, Compound (1) or a pharmaceutically acceptable salt thereof is administered within 3 hours of the onset of symptoms. In certain embodiments, Compound (1) or a pharmaceutically acceptable salt thereof is administered within 2 hours of the onset of symptoms. In certain embodiments, Compound (1) or a pharmaceutically acceptable salt thereof is administered within 1 hour of the onset of symptoms. In certain embodiments, Compound (1) or a pharmaceutically acceptable salt thereof is administered within 30 minutes of the onset of symptoms. In certain embodiments, Compound (1) or a pharmaceutically acceptable salt thereof is administered within 15 minutes of the onset of symptoms.

In certain embodiments, the symptoms are selected from chest pain or discomfort, shortness of breath, dizziness or light-headedness, nausea or vomiting, diaphoresis, palpitations, and anxiety or dread. In certain embodiments, the diagnosis of ST-segment elevation is established by characteristic ECG findings.

### Clinical Objectives

In certain embodiments of the methods described herein, treating comprises restoring coronary artery blood flow. In certain embodiments, treating comprises at least partially restoring coronary artery blood flow. For example, coronary artery blood flow may be restored by about 10%, about 20%, about 30%, about 40%, about 50%, about 60%, about 70%, about 80%, about 90%, or about 100%.

In certain embodiments of the methods described herein, treating comprises restoring, at least partially restoring, or maintaining coronary artery blood flow.

In certain embodiments of the methods described herein, treating comprises achieving an improvement of Thrombolysis in Myocardial Infarction (TIMI) Frame Count, TIMI Flow Grade, or TIMI Myocardial Perfusion Grade in the subject. In certain embodiments, the improvement is relative to a placebo. Placebo refers to the absence of any treatment, or comparison to a treatment that has no biological effect. In certain embodiments, the improvement is achieved within 1-5 hours following administration of Compound (1), a pharmaceutically acceptable salt thereof, or a pharmaceutical composition as described herein.

In certain embodiments, a TIMI Frame Count reduction of at least 5 is achieved within 1-5 hours following administration of Compound (1), a pharmaceutically acceptable salt thereof, or a pharmaceutical composition as described herein.

In certain embodiments of the methods described herein, treating comprises achieving and/or maintaining in the subject a stable platelet count. In certain embodiments, the platelet count does not change by more than 10%, 20%, or 30%.

In certain embodiments of the methods described herein, treating comprises achieving ≥80% inhibition of platelet aggregation in the subject. In certain embodiments, achieving ≥80% inhibition of platelet aggregation in the subject is achieved within 5-90, 5-60, or 10-30 minutes following administration of Compound (1), a pharmaceutically acceptable salt thereof, or a pharmaceutical composition as described herein. In certain embodiments, achieving ≥80% inhibition of platelet aggregation in the subject is achieved within about 15 minutes following administration of Compound (1), a pharmaceutically acceptable salt thereof, or a pharmaceutical composition as described herein.

In certain embodiments of the methods described herein, treating comprises achieving ST deviation resolution in the subject. In certain embodiments, the ST deviation resolution in the subject is ≥70% compared to baseline. In certain embodiments, the ST deviation resolution is measured relative to placebo. In certain embodiments, the ST deviation resolution in the subject is ≥80% compared to baseline. In certain embodiments, the ST deviation resolution in the subject is ≥90% compared to baseline. In certain embodiments, ST deviation resolution in the subject is achieved within about 1-5 hours following administration of Compound (1), a pharmaceutically acceptable salt thereof, or a pharmaceutical composition as described herein.

In certain embodiments of the methods described herein, treating comprises achieving a reduction in residual ST deviation in the subject. In certain embodiments, the reduction is measured relative to placebo. In certain embodiments, a reduction in residual ST deviation in the subject is achieved following a single administration (e.g., a single subcutaneous injection) of Compound (1), a pharmaceutically acceptable salt thereof, or a pharmaceutical composition as described herein. In certain embodiments, a reduction in residual ST deviation in the subject is achieved within about 1-5 hours following administration of Compound (1), a pharmaceutically acceptable salt thereof, or a pharmaceutical composition as described herein.

In certain embodiments of the methods described herein, treating comprises achieving a reduction in all-cause mortality and/or cardiovascular mortality versus placebo at 12 months.

In certain embodiments of the methods described herein, treating comprises achieving an improvement composite of all cause death, recurrent myocardial infarction (MI), urgent target vessel revascularization (TVR), or blinded bail-out use of intravenous (IV) αIIbβ3 antagonists or IV P2Y12 antagonist in the subject up to a month following administration (e.g., a single subcutaneous injection) of Compound (1), a pharmaceutically acceptable salt thereof, or a pharmaceutical composition as described herein. In certain embodiments, the determination is made versus placebo up to 1 month post-PCI/angiography relative to placebo.

In certain embodiments of the methods described herein, treating comprises achieving reduced acute stent thrombosis in the subject up to 24 hours following administration (e.g., a single subcutaneous injection) of Compound (1), a pharmaceutically acceptable salt thereof, or a pharmaceutical composition as described herein. In certain embodiments, the reduced stent thrombosis is measured versus placebo.

In certain embodiments of the methods described herein, treating comprises achieving reduced heart failure, directly or indirectly, for several years following administration (e.g., a single subcutaneous injection) of Compound (1), a pharmaceutically acceptable salt thereof, or a pharmaceutical composition as described herein. In certain embodiments, the reduction is measured versus placebo. This effect can be predicted by measurement of B-type-natriuretic protein (BNP), specifically measuring N-terminal pro-B-Type natriuretic protein measured within about 1 day following the administration of Compound (1), a pharmaceutically acceptable salt thereof, or a pharmaceutical composition as described herein.

In certain embodiments of the methods described herein, treating comprises achieving no occurrence of death in the subject within 30 days following administration (e.g., a single subcutaneous injection) of Compound (1), a pharmaceutically acceptable salt thereof, or a pharmaceutical composition as described herein. In certain embodiments, the no occurrence of death is measured versus placebo.

In certain embodiments of the methods described herein, treating comprises achieving no occurrence of stroke in the subject within 30 days following administration (e.g., a single subcutaneous injection) of Compound (1), a pharmaceutically acceptable salt thereof, or a pharmaceutical composition as described herein. In certain embodiments, the no occurrence of stroke is measured versus placebo.

In certain embodiments of the methods described herein, treating comprises achieving no recurrence of myocardial infarction (MI) in the subject within 30 days following administration (e.g., a single subcutaneous injection) of Compound (1), a pharmaceutically acceptable salt thereof, or a pharmaceutical composition as described herein. In certain embodiments, the no recurrence of myocardial infarction is measured versus placebo.

In certain embodiments of the methods described herein, treating comprises achieving no occurrence of acute stent thrombosis in the subject up to 24 hours following administration (e.g., a single subcutaneous injection) of Compound (1), a pharmaceutically acceptable salt thereof, or a pharmaceutical composition as described herein. In certain embodiments, the no occurrence of acute stent thrombosis is measured versus placebo.

In certain embodiments of the methods described herein, treating comprises achieving no new onset of heart failure or rehospitalization for heart failure within 30 days following administration (e.g., a single subcutaneous injection) of Compound (1), a pharmaceutically acceptable salt thereof, or a pharmaceutical composition as described herein. In certain embodiments, the no new onset of heart failure or rehospitalization for heart failure is measured versus placebo.

In certain embodiments of the methods described herein, treating comprises achieving peak high-sensitive cardiac troponin T (hs-cTnT) less than ten times an upper limit of normal at 24 hours following administration (e.g., a single subcutaneous injection) of Compound (1), a pharmaceutically acceptable salt thereof, or a pharmaceutical composition as described herein. In certain embodiments, the peak hs-CTnT is measured versus placebo.

### Patient Populations

In certain embodiments, the subject has a history of coronary artery disease. In certain embodiments, the subject has stable coronary artery disease. In certain embodiments, the subject has no history of coronary artery disease.

In certain embodiments, the subject is being administered (or is self-administering) another pharmacological therapy at the time of onset of symptoms of the thrombotic disorder. 1s In some embodiments, the other pharmacological therapy comprises one or more of aspirin, warfarin, a statin, and a P2Y12 inhibitor.

### Particular Embodiments

In a particular aspect, provided herein is a method for treating STEMI, comprising administering by subcutaneous injection to a subject in need thereof a composition comprising 0.075 mg/kg of Compound (1). In a particular embodiment, the composition further comprises acetic acid, hydrochloric acid, glycerin, and water. In a particular embodiment, the composition further comprises acetic acid, hydrochloric acid, citric acid, glycerin, and water.

In another particular aspect, provided herein is a method for treating STEMI, comprising administering by subcutaneous injection to a subject in need thereof a composition comprising 0.09 mg/kg of Compound (1). In a particular embodiment, the composition further comprises acetic acid, hydrochloric acid, glycerin, and water. In a particular embodiment, the composition further comprises acetic acid, hydrochloric acid, citric acid, glycerin, and water.

In a particular aspect, provided herein is a method for treating STEMI, comprising administering by subcutaneous injection to a subject in need thereof a composition comprising 0.11 mg/kg of Compound (1). In a particular embodiment, the composition further comprises acetic acid, hydrochloric acid, glycerin, and water. In a particular embodiment, the composition further comprises acetic acid, hydrochloric acid, citric acid, glycerin, and water.

In a particular aspect, provided herein is a method for treating STEMI, comprising administering by subcutaneous injection to a subject in need a composition comprising 0.13 mg/kg of Compound (1). In a particular embodiment, the composition further comprises acetic acid, hydrochloric acid, glycerin, and water. In a particular embodiment, the composition further comprises acetic acid, hydrochloric acid, citric acid, glycerin, and water.

### Definitions

Unless otherwise specified, the terms “administer,” “administering,” or “administration” refers to implanting, absorbing, ingesting, injecting, inhaling, or otherwise introducing a compound described herein, or a composition thereof, in or on a subject.

The terms “condition,” “disease,” and “disorder” may be used interchangeably.

An “effective amount” of a compound, or a pharmaceutically acceptable salt thereof, described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

The term “pharmaceutically acceptable salt” refers to those salts which are, within the scope of sound medical judgment, suitable for use in contact with the tissues of humans and non-human animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The terms “treatment,” “treat,” and “treating” refer to reversing, alleviating, or inhibiting the progress of a disease or condition described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

### Compositions

In another aspect, provided herein is a composition comprising Compound (I), or a pharmaceutically acceptable salt thereof, in an amount and/or concentration as described herein, and one or more carriers, diluents, or excipients.

### EXAMPLES

### Example 1: Clinical Study Objectives and Endpoints

| Objectives | Endpoints |
|---|---|
| Primary Efficacy | |
| To assess restoration of the coronary artery | As assessed by an independent Core |
| blood flow (corrected TIMI Frame Count) | Laboratory: Corrected TIMI Frame Count |
| before PCI (or coronary angiography in | before PCI/angiography. |
| case no PCI is performed) after a single | |
| subcutaneous injection of RUC-4 versus | |
| placebo in STEMI subjects in the | |
| ambulance (pre-hospital setting). | |
| To assess resolution of ST segment | As assessed by an independent Core |
| deviation post-PCI/angiography after a | Laboratory: ST segment deviation resolution |
| single subcutaneous injection of RUC-4 | 1-hour post-PCI/angiography. |
| versus placebo in STEMI subjects in the | |
| ambulance (pre-hospital setting). | |
| Primary Safety | |
| To assess bleeding events (according to | Subject incidence of bleeding events |
| Global Use of Strategies to Open Occluded | (according to GUSTO severe criterion for |
| Coronary Arteries [GUSTO] severe | safety assessment and according to the |
| criterion for safety assessment and | BARC 3C and 5 criteria for information |
| according to the BARC 3C and 5 criteria for | only) at baseline and up to 1-month post- |
| information only) after a single | PCI/angiography. |
| subcutaneous injection of RUC-4 versus | |
| placebo up to 1 month post- | |
| PCI/angiography. | |
| Secondary | |
| Safety | |
| To assess platelet count after a single | Platelet count before PCI/angiography, at |
| subcutaneous injection of RUC-4 versus | the end of the PCI/angiography, 6 hours |
| placebo before PCI/angiography, at the end | post-PCI/angiography, 24 hours post- |
| of the PCI/CAG, 6 hours post- | PCI/angiography and at hospital |
| PCI/angiography, 24 hours post- | discharge/72-hours post-PCI/angiography |
| PCI/angiography and at hospital | (whichever occurs first). |
| discharge/72-hours post-PCI/angiography | |
| (whichever occurs first). | |
| To assess bleeding events (according to | Subject incidence of bleeding events |
| International Society on Thrombosis and | (according to ISTH Major and TIMI Major |
| Haemostasis [ISTH] Major and TIMI Major | for information only) at baseline and up to |
| for information only) after a single | 1month post-PCI/angiography. |
| subcutaneous injection of RUC-4 up to | |
| 1 month post-PCI/angiography | |
| To assess the injection site reactions of a | Subject incidence of injection site reactions |
| single subcutaneous injection of RUC-4 | at baseline, 1-hour post-PCI/angiography, |
| versus placebo at baseline, 1-hour post- | hospital discharge/72-hours post- |
| PCI/angiography, hospital discharge/72- | PCI/angiography, and at 1-month |
| hours post-PCI/angiography, and at 1-month | follow-up. |
| follow-up. | |
| Efficacy | |
| To assess a composite of all cause death, | Time in days to a composite of all cause |
| recurrent MI, urgent TVR or blinded bail- | death, recurrent MI, urgent TVR or bail- |
| out* use of IV αIIbβ3 antagonists or | out* use of IV αIIbβ3 antagonists up to |
| intravenous P2Y12 antagonist after a single | 1 month post-PCI-angiography. |
| subcutaneous injection of RUC-4 versus | |
| placebo up to 1month post-PCI- | |
| angiography. | |
| To assess acute stent thrombosis after a | Subject incidence of acute stent thrombosis |
| single subcutaneous injection of RUC-4 | up to 24 hours post-PCI (according to the |
| versus placebo up to 24 hours post-PCI. | Academic Research Consortium [ARC] |
| | definition). |
| Exploratory | |
| Procedural (Angiographic) | |
| To assess TIMI thrombus grade after a | Subject incidence of TIMI thrombus grade |
| single subcutaneous injection of RUC-4 | at first angiography. |
| versus placebo at first angiography. | |
| To assess intraprocedural thrombosis after a | Subject incidence of intraprocedural |
| single subcutaneous injection of RUC-4 | thrombosis. |
| versus placebo. | |
| To assess the incidence of TIMI grade 2 or | Subject incidence of TIMI grade 2 or 3 |
| 3 epicardial flow and TIMI grade 3 | epicardial flow and TIMI grade 3 |
| myocardial perfusion in the culprit artery | myocardial perfusion in the culprit artery |
| after a single subcutaneous injection of | before and after primary PCI/angiography. |
| RUC-4 versus placebo before and after | |
| primary PCI/angiography. | |
| ECG | |
| To assess the incidence of complete ST | Subject incidence of complete ST deviation |
| deviation resolution (≥70% compared with | resolution (≥70% compared with baseline) |
| baseline as assessed in a blinded | at 1 hour post-PCI/angiography. |
| electrocardiogramare Laboratory) after | |
| a single subcutaneous injection of RUC-4 | |
| versus placebo at 1 hour post- | |
| PCI/angiography. | |
| To assess residual ST deviation after a | Residual ST deviation on the single ECG at |
| single subcutaneous injection of RUC-4 | 1 hour post-PCI/angiography. |
| versus placebo on the single ECG at 1 hour | |
| post-PCI/angiography. | |
| To assess the incidence of Q waves after a | Subject incidence of Q waves on the 12-lead |
| single subcutaneous injection of RUC-4 | ECG at hospital discharge/72-hours post- |
| versus placebo on the 12-lead ECG at | PCI/angiography (whichever occurs first). |
| hospital discharge/72-hours post- | |
| PCI/angiography (whichever occurs first). | |
| To assess resolution of ST segment | ST segment deviation resolution pre- |
| deviation after a single subcutaneous | PCI/angiography. |
| injection of RUC-4 versus placebo pre- | |
| PCI/angiography. | |
| Biomarkers | |
| To assess high-sensitive cardiac troponin T | Hs-cTnT at 24 hours and at hospital |
| (hs-cTnT) after a single subcutaneous | discharge/72-hours post-PCI/angiography |
| injection of RUC-4 versus placebo at 24 | (whichever occurs first). |
| hours post-PCI/angiography and at hospital | |
| discharge/72-hours post-PCI/angiography | |
| (whichever occurs first). | |
| To assess N-terminal pro-B-Type | NT-proBNP levels at 24 hours post- |
| natriuretic protein (NT-proBNP) levels after | PCI/angiography. |
| a single subcutaneous injection of RUC-4 | |
| versus placebo at 24 hours post- | |
| PCI/angiography. | |
| Efficacy | |
| To assess a composite of all cause death, | Time in days to a composite of all cause |
| recurrent MI and all cause stroke (ischemic | death, recurrent MI and all cause stroke |
| and hemorrhagic bleeding) after a single | (ischemic and hemorrhagic bleeding) at 1 |
| subcutaneous injection of RUC-4 versus | month post-PCI/angiography. |
| placebo at 1 month post-PCI/angiography. | |
| To assess 1-month and 12-month mortality | Subject incidence and time to 1-month and |
| after a single subcutaneous injection of | 12-month mortality |
| RUC-4 versus placebo: | All-cause mortality |
| All-cause mortality | Cardiovascular mortality |
| Cardiovascular mortality | |
| To assess recurrent MI after a single | Subject incidence of recurrent MI at 1- |
| subcutaneous injection of RUC-4 versus | month post-PCI/angiography. |
| placebo at 1 month post-PCI/angiography. | |
| To assess urgent target lesion | Subject incidence of urgent TLR up to |
| revascularization (TLR) after a single | 1month post-PCI/angiography. |
| subcutaneous injection of RUC-4 versus | |
| placebo up to 1 month post- | |
| PCI/angiography | |
| To assess blinded thrombotic bail-out use of | Subject incidence of blinded thrombotic |
| IV αIIbβ3 or IV P2Y12 antagonists after a | bail-out use of IV αIIbβ3 or IV P2Y12 |
| single subcutaneous injection of RUC-4 | antagonists up to 24 hours post- |
| versus placebo up to 24 hours post- | PCI/angiography. |
| PCI/angiography. | |
| To assess ischemic stroke after a single | Subject incidence of ischemic stroke at 1- |
| subcutaneous injection of RUC-4 versus | month post-PCI/angiography. |
| placebo at 1-month post-PCI/angiography. | |
| To assess hospitalization for heart failure | Subject incidence of hospitalization for |
| after a single subcutaneous injection of | heart failure at 1-month post- |
| RUC-4 versus placebo at 1-month post- | PCI/angiography. |
| PCI/angiography. | |
| To assess hospitalization for atrial | Subject incidence of hospitalization for |
| fibrillation after a single subcutaneous | atrial fibrillation at 1-month post- |
| injection of RUC-4 versus placebo at | PCI/angiography. |
| 1-month post-PCI/angiography. | |
| Safety | |
| To assess safety after a single subcutaneous | Safety endpoints at 24 hours post- |
| injection of RUC-4 versus placebo | PCI/angiography and at 72-hours post- |
| throughout the study. | PCI/angiography or hospital discharge are |
| | defined as effects related to antagonism of |
| | the αIIbβ3 receptor: |
| | Subject incidence of packed red blood |
| | cell transfusions (≥2) |
| | Subject incidence of platelet |
| | transfusions |
| | Subject incidence of unplanned |
| | discontinuation of antiplatelet therapy |
| | Subject incidence of unplanned |
| | discontinuation of anticoagulant |
| | therapy |
| | Subject incidence of |
| | thrombocytopenia |
| | Subject incidence of hemorrhagic |
| | stroke at 1-month |
| | Other safety parameters: |
| | Recording of AEs, SAEs AEs up to |
| | 1 month post-PCI/angiography |
| | Laboratory parameters at baseline and |
| | at 24 hours post-PCI/angiography and |
| | at 72 hours post-PCI/angiography or |
| | hospital discharge |

*αIIbβ3 antagonists given to treat the following indications will be considered as bail-out use of αIIbβ3 antagonists: decrease in TIMI flow grade (TIMI flow grades of 0-2 or slow reflow), dissection with decreased flow, distal embolization, side-branch closure, abrupt closure of the culprit vessel, clinical instability or prolonged ischemia.

### Example 2. Revised Clinical Study Objectives and Endpoints

| Objectives | Endpoints |
|---|---|
| Primary | |
| To assess 30 day all-cause mortality after a | Subject incidence and time to 30 day all- |
| single subcutaneous injection of RUC-4 | cause mortality |
| versus placebo. | |
| To assess ischemic stroke after a single | Subject incidence of ischemic stroke at 30 |
| subcutaneous injection of RUC-4 versus | days post-PCI/angiography. |
| placebo at 30 days post-PCI/angiography. | |
| To assess recurrent MI after a single | Subject incidence of recurrent MI at 30 days |
| subcutaneous injection of RUC-4 versus | post-PCI/angiography. |
| placebo at 30 days post-PCI/angiography. | |
| To assess acute stent thrombosis after a | Subject incidence of acute stent thrombosis |
| single subcutaneous injection of RUC-4 | up to 24 hours post-PCI (according to the |
| versus placebo up to 24 hours post-PCI. | Academic Research Consortium [ARC] |
| | definition). |
| To assess new onset heart failure or | Subject incidence of new onset heart failure |
| rehospitalization for heart failure versus | or rehospitalization for heart failure at 30 |
| placebo at 30 days | days post-PCI/angiography |
| To assess high-sensitive cardiac troponin T | Subject incidence of hs-cTnT greater than |
| (hs-cTnT) after a single subcutaneous | ten times an upper limit of normal at 24 |
| injection of RUC-4 versus placebo at 24 | hours post PCI-angiography. |
| hours post-PCI/angiography. | |
| To assess absence of the above objectives at | Subject incidence of none of the above |
| their specified times. | endpoints at their specified times. |
| Secondary | |

### Example 3. In Vitro Studies

Whole blood anticoagulated with either sodium citrate (0.32%) or PPACK (100 PM) from healthy volunteers who were either taking or not taking daily aspirin was treated in vitro with Compound (I) (RUC-4) to achieve final whole blood concentrations between 30 and 900 nM. Blood was incubated for 10 minutes at room temperature and then tested with either the IIb/IIIa or PRU cartridge.

### Example 4. Study Results

### In Vitro Study Results

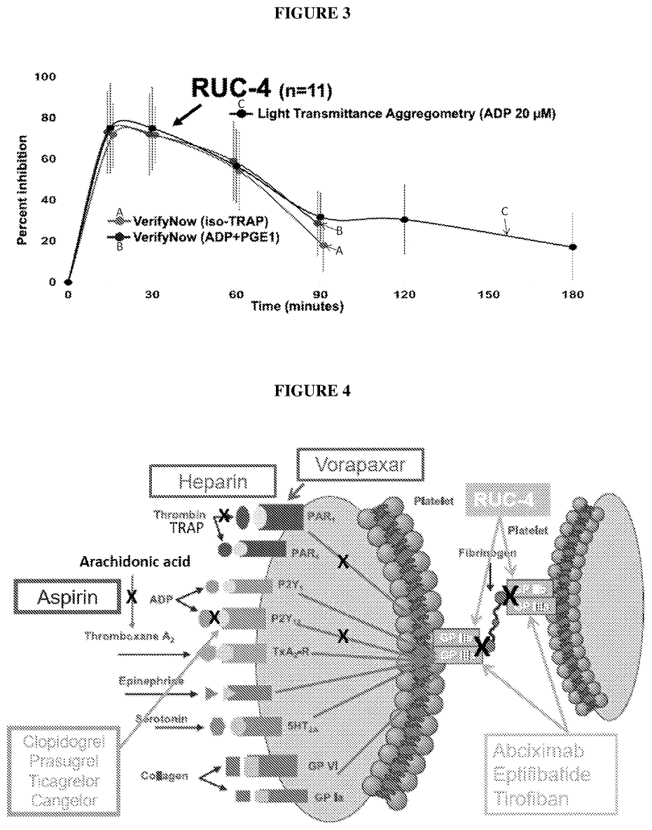

Compound (I) (RUC-4) produces dose-dependent inhibition of the VN isoTRAP channels in both the IIb/IIIa and PRU cartridges, as well as the ADP+PGE₁ channel in the PRU cartridge. Both channels give comparable results to unhibition of 20 μM ADP-Induced Light Transmission Aggregation (LTA). Since P2Y12 antagonists inhibit the ADP+PGE₁ channel, but not the isoTRAP channels, the effect of RUC-4 on patients who are not treated with a P2Y12 antagonist may be monitored with the PRU ADP+PGE₁ channel or the isoTRAP channel in either the PRU or IIb/IIIa cartridge. The RUC-4 effects in patients treated with a P2Y12 antagonist may be monitored with the isoTRAP channel in either cartridge. The results are shown in FIG. 3.

### Example 5. In Vivo Studies in Healthy Volunteers and Stable CAD Patients Taking Aspirin

Sequentially, healthy volunteers (HV) and then patients with stable coronary disease (CAD) receiving aspirin received escalating doses of RUC-4 or placebo in a sentinel dose, randomized, blinded fashion. Inhibition of platelet aggregation (IPA) to adenosine diphosphate (ADP; 20 μM), RUC-4 blood levels, laboratory evaluations, and clinical assessments were made through 24 hours and at 7 days. Doses were increased until a dose that produced ≥80% IPA within 15 minutes, with return toward baseline within 4 hours was achieved. VerifyNow (VN) was also measured in a subset of patients with stable CAD.

### Example 6. Study Results

### In Vivo Study Results

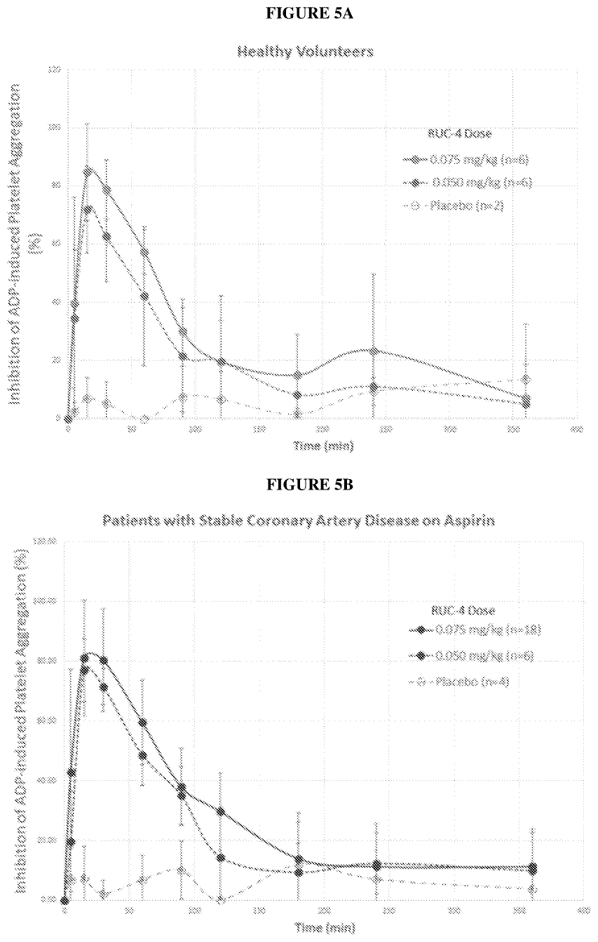

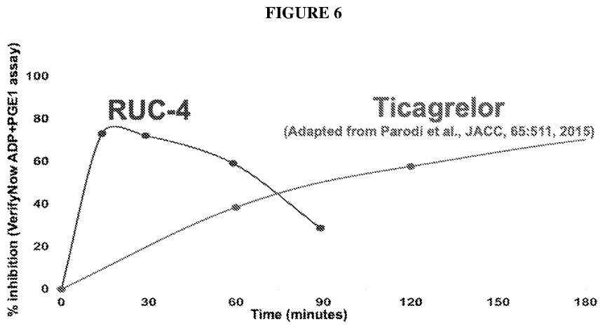

15 minutes after SC injection, mean±SD IPA was 6.9+7.1% after placebo and 71.8±15.0% at 0.05 mg/kg (n=6) and 84.7±16.7% at 0.075 mg/kg (n=6) after RUC-4. In all cases, IPA diminished over 90-120 minutes. CAD: 15 minutes after SC injection of placebo or 0.04 mg/kg (n=2), 0.05 mg/kg (n=6), and 0.075 mg/kg (n=18) of RUC-4, IPA was 14.6+11.7%, 53.6±17.0%, 76.9±10.6%, and 88.9±12.7%, respectively. RUC-4 blood levels correlated closely with IPA. Aspirin did not affect the IPA or RUC-4 blood levels. The results are shown in FIG. 5.

### Example 7. In Vivo Studies in Patients with ST-Elevation Myocardial Infarction (STEMI)

The open-label, dose escalating, phase 2, CEL-02 trial set out to assess the pharmacodynamics (PD), pharmacokinetics (PK), safety and tolerability of RUC-4 in STEMI patients treated with aspirin and ticagrelor in the ambulance undergoing primary percutaneous coronary intervention (pPCI). STEMI patients presenting for pPCI at the St. Antonius Hospital Nieuwegein, the Netherlands, received a single, weight-adjusted, subcutaneous injection of RUC-4 before coronary angiography. After assessment of every 8 evaluable patients, the dose was escalated to reach an optimal dose for platelet inhibition. All patients were concomitantly treated with aspirin, ticagrelor and unfractionated heparin (UFH)(5000 IU) in the ambulance. Additional UFH during PCI was only administered when ACT was <200 seconds (2500-5000 IU). Blood samples for (PD) and (PK) were taken before RUC-4 administration, and 15, 45, 60, 90, 120, 180, and 240 minutes thereafter. Platelet inhibition was measured by the VerifyNow P2Y₁₂ assay BASE channel which uses a protease activated receptor-1 (PAR-1) peptide (iso-TRAP) and a PAR-4 activating peptide as agonists, overriding platelet inhibition by oral P2Y₁₂ inhibitors.

### Example 8. Study Results

### In Vivo Study Results

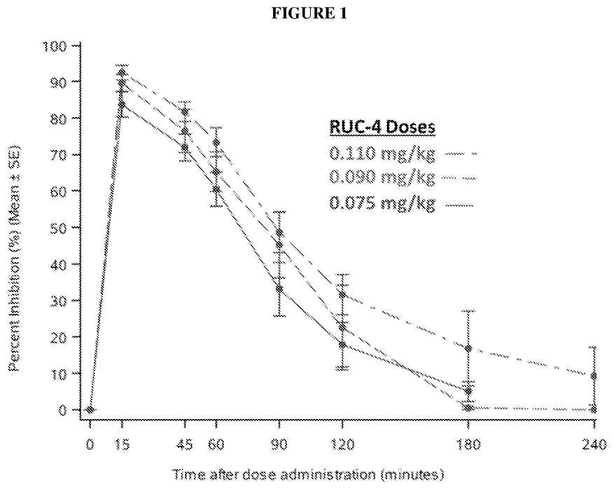

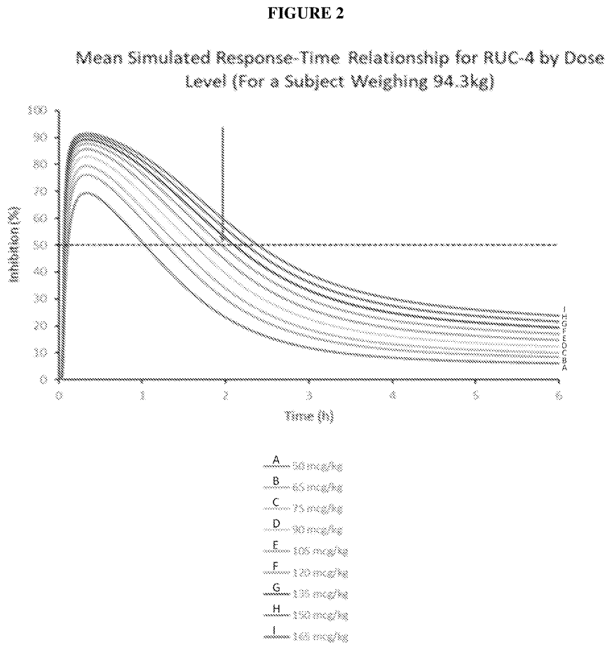

Twenty-seven patients received RUC-4 in a dose of 0.075 mg/kg (n=8), 0.090 mg/kg (n=9) or 0.11 mg/kg (n=10). RUC-4 showed dose-responsive maximal platelet inhibition at 15 minutes post-dose (mean [SD] 83.7% [9.9%], 89.6% [6.6%], and 92.5% [5.8%], respectively). At 120 minutes post-dose platelet inhibition was 17.9% (17%), 22.6% (32.9%) and 31.6% (15.7%), respectively. The results are also depicted in FIG. 1. In this study in patients with STEMI, single-dose subcutaneous RUC-4 induced a dose-dependent response of maximal platelet function inhibition within 15 minutes and a dose-dependent return of platelet function within 120 minutes.

### Embodiments

1. A method for treating or preventing a thrombotic disorder, comprising administering to a subject in need thereof an effective amount of the Compound (1):

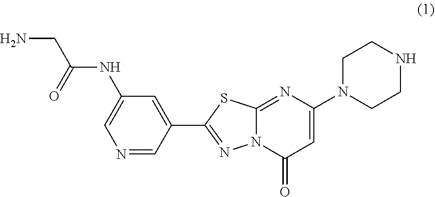 [SMILES: NCC(=O)Nc1cncc(-c2nn3c(=O)cc(N4CCNCC4)nc3s2)c1]

or a pharmaceutically acceptable salt thereof, at a dose in the range of 0.01-0.3 mg/kg, inclusive.
2. The method of embodiment 1, wherein the dose is in the range of 0.05-0.25 mg/kg, inclusive.
3. The method of any one of the preceding embodiments, wherein the dose is in the range of 0.075-0.15 mg/kg, inclusive.
4. The method of any one of the preceding embodiments, wherein the dose is in the range of 0.09-0.15 mg/kg, inclusive.
5. The method of any one of the preceding embodiments, wherein the dose is in the range of 0.1-0.15 mg/kg, inclusive.
6. The method of any one of the preceding embodiments, wherein the thrombotic disorder is myocardial infarction.
7. The method of any one of the preceding embodiments, wherein the thrombotic disorder is ST-elevated myocardial infarction (STEMI).
8. The method of any one of the preceding embodiments, wherein the compound, or pharmaceutically acceptable salt thereof, is administered by injection.
9. The method of embodiment 8, wherein the injection is intramuscular injection.
10. The method of embodiment 8, wherein the injection is subcutaneous injection.
11. The method of any one of embodiments 8-10, wherein the wherein the compound, or pharmaceutically acceptable salt thereof, is administered in the form of a solution, suspension, or emulsion.
12. The method of embodiment 11, wherein the wherein the compound, or pharmaceutically acceptable salt thereof, is administered in the form of a solution.
13. The method of embodiment 12, wherein the concentration of the compound, or pharmaceutically acceptable salt thereof, in the solution is 0.01-100 mg/mL, inclusive.
14. The method of embodiment 13, wherein the concentration is about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, or about 25 mg/mL.
15. The method of any one of embodiments 11-12, wherein the solution further comprises one or more of acetic acid, hydrochloric acid, citric acid, glycerin, and water.
16. The method of any one of embodiments 8-15, wherein administration of the compound, or pharmaceutically acceptable salt thereof, results in minimal injection site reaction in the subject at t=0, t=1 hour, t=1 day, t=3 days, or t=1 month post injection.
17. The method of any one of the preceding embodiments, wherein the subject experiences one or more symptoms of the thrombotic disorder, and the compound, or pharmaceutically acceptable salt thereof, is administered within 30 minutes to 6 hours of the onset of symptoms.
18. The method of embodiment 17, wherein the thrombotic disorder is STEMI, and the symptoms are selected from chest pain or discomfort, shortness of breath, dizziness or light-headedness, nausea or vomiting, diaphoresis, palpitations, and anxiety or dread.
19. The method of any one of the preceding embodiments, wherein treating comprises at least partially restoring, or maintaining, coronary artery blood flow.
20. The method of any one of the preceding embodiments, wherein treating comprises achieving an improvement of Thrombolysis in Myocardial Infarction (TIMI) Frame Count or TIMI flow, preferably within 1-5 hours following administration of the compound, or pharmaceutically acceptable salt thereof.
21. The method of embodiment 20, wherein treating comprises achieving an improvement of TIMI Frame Count or TIMI flow of at least 5, preferably within 1-5 hours following administration of the compound, or pharmaceutically acceptable salt thereof.
22. The method any one of the preceding embodiments, wherein treating comprises maintain a stable platelet count, preferably within 1-5 hours following administration of the compound, or pharmaceutically acceptable salt thereof.
23. The method of any one of the preceding embodiments, wherein treating comprises achieving ≥80% inhibition of platelet aggregation, preferably within 5-90 minutes (e.g., 15 minutes) following administration of the compound, or pharmaceutically acceptable salt thereof.
24. The method of any one of the preceding embodiments, wherein treating comprises achieving improved ST deviation resolution (≥70% compared to baseline), preferably within 1-5 hours following administration of the compound, or pharmaceutically acceptable salt thereof.
25. The method of any one of the preceding embodiments, wherein treating comprises achieving a reduction in residual ST deviation after a single subcutaneous injection.
26. The method of any one of the preceding embodiments, wherein treating comprises achieving an improvement composite of all cause death, recurrent myocardial infarction (MI), urgent target vessel revascularization (TVR) or blinded bail-out use of intravenous (IV) αIIbβ3 antagonists or IV P2Y12 antagonist after a single subcutaneous injection.
27. The method of any one of the preceding embodiments, wherein treating comprises achieving reduced acute stent thrombosis after a single subcutaneous injection.
28. The method of any one of the preceding embodiments, wherein treating comprises achieving no occurrence of death in the subject within 30 days following administration of the compound, or pharmaceutically acceptable salt thereof.
29. The method of any one of the preceding embodiments, wherein treating comprises achieving no occurrence of stroke in the subject within 30 days following administration of the compound, or pharmaceutically acceptable salt thereof.
30. The method of any one of the preceding embodiments, wherein treating comprises achieving no recurrence of myocardial infarction (MI) in the subject within 30 days following administration of the compound, or pharmaceutically acceptable salt thereof.
31. The method of any one of the preceding embodiments, wherein treating comprises achieving no acute stent thrombosis in the subject up to 24 hours following administration of the compound, or pharmaceutically acceptable salt thereof.
32. The method of any one of the preceding embodiments, wherein treating comprises achieving no new onset of heart failure or rehospitalization for heart failure in the subject within 30 days following administration of the compound, or pharmaceutically acceptable salt thereof.
33. The method of any one of the preceding embodiments, wherein treating comprises achieving peak high-sensitive cardiac troponin T (hs-cTnT) less than ten times an upper limit of normal at 24 hours following administration of the compound, or pharmaceutically acceptable salt thereof.
34. The method of any one of the preceding embodiments, further comprising administering a second dose of Compound (1), or pharmaceutically acceptable salt thereof.
35. The method of any one of the preceding embodiments, further comprising administering to the subject a P2Y12 inhibitor, e.g., ticagelor, prasugrel, or clopidogrel.
36. The method of embodiment 34, further comprising administering to the subject aspirin.
37. The method of embodiment 35, comprising administering to the subject aspirin and a P2Y12 inhibitor.
38. The method of embodiment 35, comprising administering to the subject aspirin, a P2Y12 inhibitor, and heparin.
39. The method of any one of the preceding embodiments, wherein the subject has been administered aspirin, e.g., prior to the onset of symptoms, or after the onset of symptoms but prior to administration.
40. The method of embodiment 34, further comprising administering to the subject heparin.
41. The method of any one of the preceding embodiments, wherein the subject has a history of coronary artery disease.
42. The method of any one of the preceding embodiments, wherein the subject has stable coronary artery disease.

1. A method for treating or preventing a thrombotic disorder, comprising administering to a subject in need thereof an effective amount of the Compound (1): 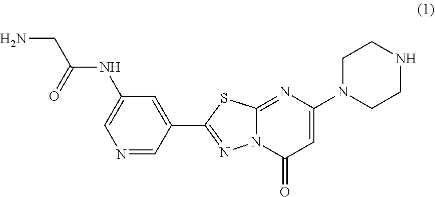 [SMILES: NCC(=O)Nc1cncc(-c2nn3c(=O)cc(N4CCNCC4)nc3s2)c1]
or a pharmaceutically acceptable salt thereof, at a dose in the range of 0.01-0.3 mg/kg, inclusive.

2-5. (canceled)

6. The method of claim 1, wherein the thrombotic disorder is myocardial infarction or ST-elevated myocardial infarction (STEMI).

7. (canceled)

8. The method of claim 1, wherein the compound, or pharmaceutically acceptable salt thereof, is administered by injection.

9. (canceled)

10. (canceled)

11. The method of claim 8, wherein the wherein the compound, or pharmaceutically acceptable salt thereof, is administered in the form of a solution, suspension, or emulsion.

12-14. (canceled)

15. The method of claim 11, wherein the solution further comprises one or more of acetic acid, hydrochloric acid, citric acid, glycerin, and water.

16. The method of claim 8, wherein administration of the compound, or pharmaceutically acceptable salt thereof, results in minimal injection site reaction in the subject at t=0, t=1 hour, t=1 day, t=3 days, or t=1 month post injection.

17. The method of claim 1, wherein the subject experiences one or more symptoms of the thrombotic disorder, and the compound, or pharmaceutically acceptable salt thereof, is administered within 30 minutes to 6 hours of the onset of symptoms.

18. (canceled)

19. The method of claim 1, wherein treating comprises at least partially restoring, or maintaining, coronary artery blood flow.

20. The method of claim 1, wherein treating comprises achieving an improvement of Thrombolysis in Myocardial Infarction (TIMI) Frame Count or TIMI flow, preferably within 1-5 hours following administration of the compound, or pharmaceutically acceptable salt thereof; or wherein treating comprises achieving an improvement of TIMI Frame Count or TIMI flow of at least 5, preferably within 1-5 hours following administration of the compound, or pharmaceutically acceptable salt thereof.

21. (canceled)

22. The method of claim 1, wherein treating comprises maintaining a stable platelet count, preferably within 1-5 hours following administration of the compound, or pharmaceutically acceptable salt thereof.

23. The method of claim 1, wherein treating comprises achieving ≥80% inhibition of platelet aggregation, preferably within 5-90 minutes following administration of the compound, or pharmaceutically acceptable salt thereof.

24. The method of claim 1, wherein treating comprises achieving improved ST deviation resolution of ≥70% compared to baseline, preferably within 1-5 hours following administration of the compound, or pharmaceutically acceptable salt thereof; or wherein treating comprises achieving a reduction in residual ST deviation after a single subcutaneous injection.

25. (canceled)

26. The method of claim 1, wherein treating comprises achieving an improvement composite of all cause death, recurrent myocardial infarction (MI), urgent target vessel revascularization (TVR) or blinded bail-out use of intravenous (IV) αIIbβ3 antagonists or IV P2Y12 antagonist after a single subcutaneous injection.

27. The method of claim 1, wherein treating comprises achieving reduced acute stent thrombosis after a single subcutaneous injection, or achieving no acute stent thrombosis in the subject up to 24 hours following administration of the compound, or pharmaceutically acceptable salt thereof.

28. (canceled)

29. The method of claim 1, wherein treating comprises achieving no occurrence of stroke in the subject within 30 days following administration of the compound, or pharmaceutically acceptable salt thereof.

30. The method of claim 1, wherein treating comprises achieving no recurrence of myocardial infarction (MI), no new onset heart failure, or rehospitalization for heart failure, in the subject within 30 days following administration of the compound, or pharmaceutically acceptable salt thereof.

31. (canceled)

32. (canceled)

33. The method of claim 1, wherein treating comprises achieving peak high-sensitive cardiac troponin T (hs-cTnT) less than ten times an upper limit of normal at 24 hours following administration of the compound, or pharmaceutically acceptable salt thereof.

34. (canceled)

35. The method of claim 1, further comprising administering to the subject one or more of a P2Y12 inhibitor, aspirin, and heparin.

36-38. (canceled)

39. The method of claim 1, wherein the subject has been administered aspirin prior to the onset of symptoms, or after the onset of symptoms but prior to administration.

40. (canceled)

41. The method of claim 1, wherein the subject has a history of coronary artery disease, or the subject has stable coronary artery disease.

42. (canceled)
